(19) United States
(12) Patent Application Publication
(10) Pub. No.: US 2006/0115459 A1
(43) Pub. Date: Jun. 1, 2006

(54) Sickle cell anemia treatment

(76) Inventor: Renjit Sundharadas, Milwaukee, WI (US)

Correspondence Address: Dr. Renjit Sundharadas, M.D., 11454 Raedene Way, San Diego, CA, 92131, US

(21) Appl. No.: 11/286,472

(22) Filed: Nov. 26, 2005

Related U.S. Application Data
(60) Provisional application No. 60/631,225, filed on Nov. 27, 2004.

(51) Int. Cl. A61K 35/14; C12N 5/08

(52) U.S. Cl. 424/93.7; 435/372

(57) ABSTRACT

This patent is an evolution of U.S. Pat. No. 4,216,770 named “sickle cell therapeutic treatment”. That patent is one in which blood is taken out of the subject and treated with anti-sickling agents which are then washed out. In this application blood is removed from a subject and instead filtered to remove diseased cells. The option to introduce anti-sickling agents is then left as a possible option. The filtered cells are then returned to the subject. This technology is superior to other treatments since it can help end sickle cell crises before permanent damage it done. Instead of sickled cells breaking down within the body where they compound damage, they are removed before they can cause problems. More deformable and useful blood cells are retained in the circulation.

### CROSS-REFERENCE TO RELATED APPLICATIONS

This application claims the benefit of U.S. Provisional Application No. 60/631,225 filed on Nov. 27^{th}, 2004 and incorporated herein by reference in its entirety.

This utility application is based on a provisional patent application filed on Nov. 27^{th} by Dr. Renjit Sundharadas entitled “Method of Treatment for Sickle Cell Disease”. Express label No. ER 983579633 U.S.

The Application No. is 60/631,225. The Confirmation number is 9251.

The Filing Receipt bar code has the following written under it:

*OC000000014841792*

This treatment for Sickle Cell Anemia uses off the shelf technology to remove diseased sickled cells from the circulatory system and thus prevent or treat sickle cell anemia. The subject's blood would first be routed to a cell separator. The red blood cells would be removed and then filtered so that the rigid, sickled cells would be caught in the filter and non-diseased cells would pass through. Then the red blood cell mass along with the rest of the blood mass would be returned to the patient. This would decrease the percentage of sickled cells and thus abort active crises while also preventing future crises.

### STATEMENT REGARDING FEDERALLY SPONSORED RESEARCH OR DEVELOPMENT

None

### REFERENCE TO SEQUENCE LISTING, A TABLE, OR A COMPUTER PROGRAM LISTING COMPACT DISC APPENDIX

None

### BACKGROUND OF THE INVENTION

A review of the current patent literature and medical literature shows various treatments for sickle cell anemia. Currently most treatments focus on medical or biological ways to prevent sickling. Currently the only successful treatment for the disease is a bone marrow transplant. Other treatments are basically supportive and none has proved completely successful. Gene therapy is well off in to the future. I have proposed a new therapy to address the removal of sickled cells from circulation to prevent and treat crises as well as long-term end organ damage.

The pathogenesis of the disease involves damage mainly caused by sickled cells causing micro vascular occlusions that can even cause macro vascular occlusions or other long-term end organ dysfunction. Complications can include stroke, osteopenia, and can often cause death. The average lifespan of a sickle cell disease (SCD) patient is decades shorter than a normal individual. Estimates are that up to over $3 billion is spent on SCD annually with much of it being palliative or to treat end organ damage or other side effects or complications. There has been no therapy aimed at preventing end organ damage by any other means other than preventing red blood cells from sickling.

In cases of crisis it has proven advantageous to replace red blood cells (RBCs) with transfused RBCs and hydration that thus decrease the percentage of sickled RBCs in the bloodstream. This is done either by simple transfusion or by erythrocytophoresis which separates out red blood cells from the blood by taking advantage of differences in sedimentation rates. It is noted that in splenic sequestration that simple transfusion of normal RBCs can actually reverse the disease process and cause release of sequestered cells, thus giving support the then theory of increasing the percentage of oxygenated cells as being therapeutic in the disease process.

This implies that normal red blood cells have a role in dislodging sickled cells. One must note that severely sickled cells lose their ability to carry oxygen and are not deformable like normal red blood cells. In patients with SCD the sickled cells can stay in the blood stream for about 10-20 or more days. These sickled cells are eliminated from the blood stream by often being broken down in end organs or getting stuck in microvasculature where they can cause end organ damage and also further cause local hypoxia which then can cause even more rapid sickling of other cells as well as tissue damage. Any method to remove the sickled cells before they can cause significant damage would be valuable in the management of the disease. A simple mechanical method to accomplish this is proposed.

### BRIEF SUMMARY OF THE INVENTION

This invention involves using off the shelf technology to remove sickled cells from the bloodstream and thus treat sickle cell anemia. This technology is very important during sickle cell crises. Blood is continuously drawn from a subject and diverted to a cell separator where the red cell blood mass is separated from the rest of the blood which is returned to the patient with or separately from treated red blood cells. In the cell separator red blood cells are separated by centrifugation since red blood cells have a unique sedimentation rate. These cells are then sent to a filtration chamber where the deformed sickle cells are removed. Before or after this chamber the cells can be treated to manipulate their sickling properties. This can be done by varying the environment the cells are exposed to as well as by adding biological and non-biological components. An example of a biological component would be donor red blood cells. Non-biological components can include drugs or environmental components such as oxygen. A wash chamber may be necessary to removed unwanted substances before the blood is returned to the subject.

All of the subcomponents of the proposed invention are well known in the field of medical science. Some of the above technology is currently employed in the field of medicine while the rest is utilized in medical research. Thus these technologies themselves are prior art but the combination of the above is a unique application.

### BRIEF DESCRIPTION OF THE SEVERAL VIEWS OF THE DRAWING

No Drawings

### DETAILED DESCRIPTION OF THE INVENTION

The following description of the invention contains a novel application of exclusively off the shelf technology that has already been rendered to practice in modern medical treatment and scientific research.

First, blood is withdrawn from the patient using common vascular access techniques and technology. This whole blood is taken from the subject and enters a cell separator. A cell separator is currently the preferred method for red blood cell donation. This separator uses a centrifuge to separate the blood components based on sedimentation rate. With the red blood cell mass removed from the whole blood using a cell separator, it is sent to a filtration chamber whereas the rest of the blood which includes white blood cells, platelets and plasma is returned to the patient.

Once the red blood cell is sent to the filtration chamber, it is diluted and filtered to remove disease red blood cells. Diseased red blood cells in sickle cell anemia are rigid as opposed to non-diseased cells which are highly deformable. Thus a non-diseased cell will be able to deform and pass through a filter hole that is actually smaller than the diameter of the red blood cell itself. On the other hand diseased red blood cells have hemoglobin that polymerizes. This polymerization as well as dehydration of the diseased red blood cell causes the cell to be rigid to the point that it can't deform and pass through the filter. Filter pore size would be optimized and variable to change filtration properties.

The above is well documented in the medical literature. This property of diseased sickle cells was discovered when sickle cell patients donated blood. Their diseased blood would clog filters used to remove white blood cells from the donated product. The filterability of sickle cells has been studied before and factors such as viscosity, ion concentration, oxygen tension, and osmolarity can be adjusted to optimize the process. Anticoagulants can be added at any step in the process as needed.

The filtration chamber would be made to handle a given load of red blood cell mass and would be replaced or blood diverted to another filter chamber since by definition every filter has a certain filtration capacity. This can either be done by running a set volume through each filter or by measuring the filtration pressure and diverting to a new filter once a certain level of pressure has been obtained. This filtration property has been implied by previous medical literature.

Multi-step filtration may be performed to make the process more efficient such that each successive filtration step removes selectively more deformable cells.

At any point in the process donor red blood cells can be added. This enhances the process since these normal cells will pass through the filter and not be wasted.

After the red blood cells have been successfully filtered, they are returned to the patient.

At any point in this cycle, anti-sickling agents may be added. There may also be a washing cycle.

Currently one treatment for sickle cell anemia is to simply add donor red blood cells. Another is to add donor red blood cells while also non-selectively removing all red blood cells from a different access point on the subject. This process selectively removes only the diseased cells and returns the rest to the patient. Thus, it is a permutation of existing treatment strategies. Since no good blood cells are wasted, this treatment can be used on all patients undergoing crises. It also decreases the need for transfusions and thus decreases the future risk of transfusion reactions. For those patients with frequent attacks, this procedure can be done to prevent crises and/or to manage pain.

What is claimed is:

1. A process for treating sickle cell disease by withdrawing whole blood substantially continuously from a subject and during said withdrawal of said whole blood performing the following steps:
directing the whole blood to a red cell separator;
separating red blood cells to be treated from said plasma by centrifugation;
directing the centrifuged separated red blood cells to be treated to a filtration chamber while returning the remaining blood components to the patient;
filtering the red blood cells in a single or multi-step process to remove diseased cells;
directing filtered red blood cells back to the patient

2. Optimizing filtration by adjusting filter pore size, dilution, oxygenation, ion concentration, viscosity, temperature, and by replacing filters as needed by monitoring filter pressure and/or filtrate flow volume and diverting to new filters as needed.

3. Adding biological (donor red blood cells) and non-biological agents at any point to optimize the process while having the option to wash excess agent out before returning red blood cells to the patient.
